(19) United States
(12) Plant Patent Application Publication
(10) Pub. No.: US 2006/0143759 P1
(43) Pub. Date: Jun. 29, 2006

(54) CHRYSANTHEMUM PLANT NAMED 'ARTIST ORANGE'

(50) CHRYSANTHEMUM MORIFOLIUM; CHRYSANTHEMUM

(75) Inventor: Mark Roland Boeder, The Hague (NL)

Correspondence Address: Charles A. Wendel;STEPTOE & JOHNSON LLP, 1330 Connecticut Avenue, N.W., Washington, DC, 20036, US

(73) Assignee: Chrysanthemum Breeders, Aalsmeer (NL)

(21) Appl. No.: 11/020,351

(22) Filed: Dec. 27, 2004

(51) Int. Cl. A01H 5/00

(52) U.S. Cl. PLT/296

(57) ABSTRACT

A Chrysanthemum plant named ‘Artist Orange’ characterized by its medium sized daisy-type blooms with yellow/red ray-florets and green disc florets.

### RELATED CULTIVARS

‘Artist Orange’ is related to ‘Artist Yellow’ (U.S. Plant Pat. No. 14,806), of which it is a color mutant.

### BACKGROUND OF THE INVENTION

‘Artist Orange.’ is a product of a breeding and selection program which had the objective of finding color mutants of ‘Artist Yellow’. The new plant of the present invention comprises a new and distinct cultivar of chrysanthemum plant that is a natural occurring sport of a parent chrysanthemum named ‘Artist Yellow’ . A comparison with parent chrysanthemum ‘Artist Yellow’ is also made in this application. The new and distinct cultivar was discovered as a whole plant mutation by Mark Roland Boeder in a controlled environment (greenhouse) in Rijsenhout Holland in 2002. The first act of asexual reproduction of ‘Artist Orange’ was accomplished when vegetative cuttings were taken from the initial selection in 2002 in a controlled environment in Rijsenhout, Holland.

### SUMMARY OF THE INVENTION

The present invention is a new and distinct variety of chrysanthemum bearing medium sized daisy blooms with orange/red ray-florets and green disc florets.

### BRIEF DESCRIPTION OF THE DRAWINGS

The present invention of a new and distinct variety of chrysanthemum is shown in the accompanying drawings, the color being as nearly true as possible with color photographs of this type.

FIG. 1 shows a plant of the cultivar in full bloom.

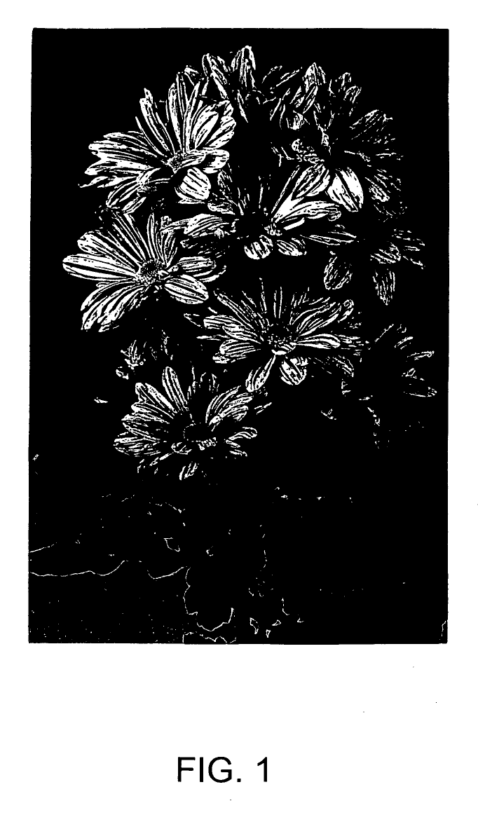

FIG. 2 shows the various stages of bloom of the new cultivar.

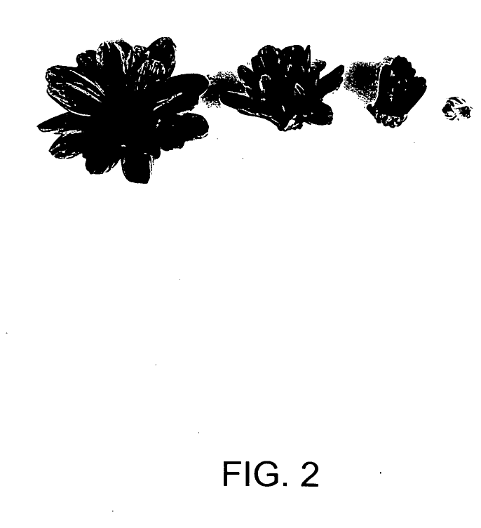

FIG. 3 shows the foliage of the new cultivar.

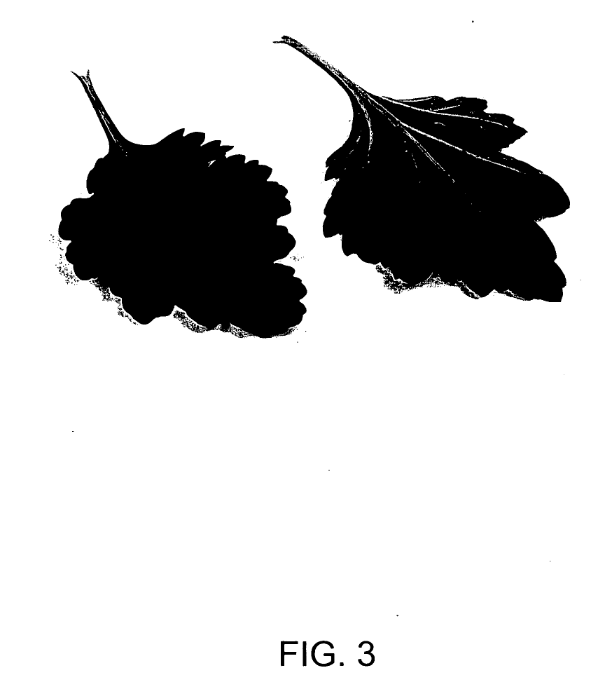

### DESCRIPTION OF THE INVENTION

This new variety of chrysanthemum is of the botanical classification Chrysanthemum morifolium L. The observations and measurements were gathered from plants grown in April/May in a greenhouse in Rijsenhout Holland in a photo-periodic controlled crop under conditions generally used in commercial practice. The greenhouse temperatures during this crop were at day-time between 18° C. and 25° C. and at night 20° C. After a long day period of 14 days the photo-periodic response time in this crop was 54 days. After the long day period to flowering plant growth retardants were applied once in a dose of 2 gram/liter water. The plants were observed (directly) during the flowering of this crop. The plant is sensitive to infestation by Powdery Mildew. The plant is tolerant to low night temperatures. This new variety produces medium sized blooms with yellow/red ray-florets and green disc-florets blooming on the plant for 5 weeks. This new variety of chrysanthemum has been found to retain its distinctive characteristics throughout successive propagations however the phenotype may vary significantly with variations in environment such as light intensity and temperature. To show the phenotype as described ‘Artist Orange’ can be planted without assimilation lightning (high pressure sodium lamps) between week 1 and week 35 under greenhouse conditions in Holland. With assimilation light (minimum level 2500 lux) it can be planted year round under greenhouse conditions in Holland.

From the cultivars known to inventor the most similar existing cultivar in comparison to ‘Artist Orange’ is ‘Artist Yellow’ and ‘Orange Reagan’ (U.S. Plant Pat. No. 8,769). When ‘Artist Orange’, ‘Artist Yellow’, and ‘Orange Reagan’ are being compared the following differences and similarities are noticed: All three plants have daisy-type blooms. The difference of ‘Artist Orange’, ‘Artist Yellow’ and ‘Orange Reagan’ is the color of the ray-florets. In ‘Orange Reagan’ this color is orange, while it is in ‘Artist Orange’ a pattern of red streaks on an orange background, and in ‘Artist Yellow’ red streaks on a yellow background.

The following is a description of the plant and characteristics that distinguish ‘Artist Orange ’ as a new and distinct variety. The color designations are taken from the plant itself. Accordingly, any discrepancies between the color designations and the colors depicted in the photographs are due to photographic tolerances. The color chart used in this description is: The Royal Horticultural Society Colour Chart, edition 1995.

TABLE 1
Botanical Description of chrysanthemum plant ‘FLORINA’

| | |
|---|---|
| Bud | |
| Size | Small; cross-section |
| | 0.5 cm, height 0.5 cm |
| Outside Color | Green 139B |
| Involucral bracts | 2 rows, length 7 mm, width 3 mm |
| Involucral bracts among disc-florets | Not present |
| Involucral bracts color | Green 139B |
| Bloom | |
| Type | Daisy |
| Height | 0.5 cm |
| Size | Small |
| Fully Expanded | 3.5 cm |
| Peduncle length | 5.5 cm |
| Peduncle color | Green 138B |
| Number of blooms per branch | Approx. 7 blooms per branch |
| Performance on the plant | 4 weeks |
| Seeds | Produced in small quantities, |
| | ovate grey-brown 199A, |
| | 1½ mm in length. |
| Fragrance | Typical chrysanthemum, |
| | slightly sweet |
| Color | |
| Center of the flower (disc-florets) | Immature Yellow-orange |
| | 14A with dot of Green |
| | 143C |
| | Mature Yellow 13A |
| Color of upper surface of the ray- | Yellow 12A |
| florets | |
| Color of the lower surface of the | Yellow 12B |
| ray-florets | |
| Tonality from Distance | A garden mum with yellow |
| | flowers and a yellow |
| | disc |
| Discoloration to color | Yellow 12A |
| Ray florets | |
| Texture | Upper and under side smooth |
| Number | 40 |
| Cross-section | Flat |
| Longitudinal axis of majority | Straight |
| Length of corolla tube | 0.3 cm |
| Ray-floret margin | Entire |
| Ray-floret length | 1.5 cm |
| Ray-floret width | 0.4 cm |
| Ratio length/width | High |
| Shape of tip | Pointed |
| Disc florets | |
| Disc diameter | 1 cm |
| Distribution of disc florets | Numerous, clearly visible |
| | at all stages of flowering |
| Shape | Tubular |
| Receptacle shape | Conical raised |
| Reproductive Organs | |
| Stamen | present in disc florets only |
| Stamen color | Yellow-green 144A |
| Pollen | Present |
| Pollen color | Yellow 7A |
| Styles | Thick |
| Style color | Yellow 13A |
| Style Length | 4 mm |
| Stigma color | Yellow-green 154D |
| Stigma Width | 1 mm |
| Ovaries | Enclosed in calyx |
| Plant | |
| Form | Grown as a spray type potmum, |
| | outdoor mounded and round |
| Growth habit | Spherical shape |
| Growth rate | High |
| Height | 25 cm |
| Width | 45 cm |
| Stem Color | Greyed-brown 199B |
| Stem Strength | Weak |
| Stem Brittleness | Not brittle |
| Stem Anthocyanin Coloration | Absent |
| Internode length | 1.5-2 cm |
| Length of lateral branch | From top to bottom 11 cm |
| Lateral branch color | Yellow-green 144A |
| Lateral branch, attachment | Medium strength |
| Branching (average number of | Prolific with 10-12 breaks |
| lateral branches) | after pinching |
| Natural season blooming date | SEPTEMBER 27- |
| | OCTOBER 3 (WEEK 40) |
| Foliage | |
| Leaf color | Upper side Green 138A-139 A |
| | Lower side Green 138A-138B |
| Color midvein | Upper side Yellow-green 147D |
| | Lower side Yellow-green 148D |
| Size | Small.; length 3-3.5 cm, |
| | width 1.7-2 cm |
| Quantity (number per lateral | 20 |
| branch) | |
| Shape | Ovate |
| Texture upper side | Glabrous |
| Texture under side | Pubescent |
| Venation arrangement | Palmate |
| Shape of the margin | Serrated |
| Shape of Base of Sinus Between | Rounded |
| Lateral Lobes | |
| Margin of Sinus Between Lateral | Diverging |
| Lobes | |
| Shape of Base | Acute |
| Apex | Mucronulate |
| Petiole lenght | 0.5 cm |
| Petiole color | Yellow-green 147D |

TABLE 2
Differences with the comparison Varieties

| | ‘Artist | ‘Artist | ‘Orange |
|---|---|---|---|
| | Orange’ | Yellow’ | Reagan’ |
| Color upper side | Orange 29A | Yellow-orange | Greyed-orange |
| ray-florets | and Red 45B | 14B and | 169A-169B |
| | | Red 46A | |
| Color lower side | Greyed-orange | Yellow-orange | Greyed-orange |
| ray-florets | 164C and | 14C and | 163B |
| | greyed-orange | Red 46B | |
| | 173B | | |

I claim:

1. A new and distinct variety of chrysanthemum plant as described and illustrated.
